(19) United States
(12) Patent Application Publication
(10) Pub. No.: US 2010/0191323 A1
(43) Pub. Date: Jul. 29, 2010

(54) BIODEGRADABLE STENT GRAFT

(76) Inventor: Mitchell Wayne Cox, Bethesda, MD (US)

Correspondence Address: BANNER & WITCOFF, LTD., 1100 13th STREET, N.W., SUITE 1200, WASHINGTON, DC, 20005-4051, US

(21) Appl. No.: 12/358,537

(22) Filed: Jan. 23, 2009

(51) Int. Cl. A61F 2/06

(52) U.S. Cl. 623/1.38

(57) ABSTRACT

A stent may help to reconstruct tissue in a vessel by causing the tissue to re-epithelialize. The stent may include a biodegradable frame and a sheet that coats the frame. The sheet may contain a biological material and may flex in unison with the frame in a radial direction. When placed in a vessel, the stent may at least partially conform to at least a portion of a vessel wall. The stent may be capable of being absorbed over a period of time, such as five years, one year, or six months. The stent may flex as the vessel wall dilates and constricts. The stent may be placed in any type of vessel including an artery and mobile vessels. The biodegradable frame may be made of a poly-lactide and/or magnesium. The sheet may contain a biological material including biologic arterial graft and/or an acellular dermal matrix.

### BACKGROUND OF THE INVENTION

1. Field of the Invention

The present invention relates to a device for tissue reconstruction. The invention concerns, more particularly, a biodegradable stent having a sheet containing a biological material that is used to reconstruct tissue in blood vessels.

2. Description of Background Art

A vessel wall may be damaged in many ways including chronic wear and weakening of the tissue that may eventually lead to weakened ability to dilate and constrict the wall of the vessel (e.g., aneurysm, artherosclerosis, angina, stroke, and other common types of ischemia). Stents are commonly used for repairing vessel walls in patients suffering from chronic and acute vessel tissue injuries. Vessels may also suffer acute trauma-related injuries such as from gunshot wounds, puncturing, bruising, or otherwise damaging the vessel wall by an object. Oftentimes, vessel damage leads to serious medical conditions and may result in long-term injury or even death of a patient.

When a vessel wall ruptures, gets punctured, cut, or otherwise damaged, blood may leak through the damaged portion of the vessel into the surrounding tissue causing significant damage. Such an injury causes blood pressure through the damaged portion of the vessel to drop and may prevent oxygenated blood from reaching a particular organ or other tissue destination or deoxygenated blood from reaching the lungs.

Endovascular surgery may offer a solution for repairing damage to the vessels and/or preventing future damage to the vessels. A may provide a reinforcement to a vessel wall to a damaged area. For example, an expandable stent, in its retracted form, may be positioned over a balloon catheter having a guide wire attached at one end. The stent may be delivered to the site of the injury. The stent may be expanded to fit the shape of the vessel wall by controlling the inflation of the balloon catheter. The stent may remain in contact with the vessel wall as a result of the radial pressure from blood flowing through the injured portion of the vessel wall. Examples of commonly known expandable stents include, U.S. Pat. No. 4,655,771 to Wallsten, U.S. Pat. No. 5,061,275 to Wallsten, et al., and U.S. Pat. No. 5,645,559 to Hachtmann, et al.

Most stents are permanently implanted into the vessel of a patient who suffered a vessel injury. Oftentimes, stents that are permanently implanted within a vessel cause complications over a length of time. This can be especially problematic with some young patients. One common complication that can occur is restonisis which can be caused by surrounding tissue reaction. In addition to the stent gradually blocking up the vessel, it is also possible that the stent can also fracture.

Endografts are similar to the stents described above but are typically placed in a larger vessels and are typically used to repair damaged tissue or improve an otherwise unhealthy portion of a vessel, which in turn is intended to prevent leakages or ruptures in a vessel wall. The endografts have a stent-like frame and are covered by a synthetic material. The outside of the synthetic material is covered with an adhesive to adhere to the inside of a vessel wall. However, during this procedure, the endograft remains in place for life commonly requiring lifetime follow up to the site.

### SUMMARY OF THE INVENTION

Although stents and stent systems exist within the art, there is room for improvement. Accordingly, a stent graft that is partially or completely biodegradable would be a welcomed advancement in the art. Also, a stent that has these capabilities and is capable of expanding across mobile vessels and vessels that extends across a joint would also be beneficial.

Aspects of the present invention involve a biodegradable stent graft for reconstructing tissue in a vessel. The stent may comprise a biodegradable frame and a sheet containing a biological material. The sheet may coat the biodegradable frame and may be capable of flexing in cooperation with the frame. The biodegradable frame and the sheet may be capable of flexing in a radial direction to at least partially conform to at least a portion of a wall of the vessel.

In another aspect of the invention, a biodegradable stent graft may comprise a biodegradable frame and a sheet that may contain a biological material. The frame may have a plurality of discrete expandable elements. The sheet may coat at least a portion of the discrete expandable elements. The sheet may also be capable of adhering to the interior surface of a wall of a vessel. The frame and the sheet may be capable of expanding in cooperative engagement with each other to adhere to the shape of the interior surface of the wall of the vessel.

In another aspect of the invention, a method of reconstructing tissue in a vessel includes positioning a delivery device at a site of damage in a vessel of a patient. The delivery device including a biodegradable frame and a sheet containing a biological material. The delivery device is affixed to a wall of the vessel at the site of damage, and is left in the vessel to permit the biodegradable frame to completely dissolve and be carried off in the blood stream.

The advantages and features of novelty characterizing aspects of the present invention are pointed out with particularity in the appended claims. To gain an improved understanding of the advantages and features of novelty, however, reference may be made to the following descriptive matter and accompanying drawings that describe and illustrate various embodiments and concepts related to the invention.

### DESCRIPTION OF THE DRAWINGS

The foregoing Summary of the Invention, as well as the following Detailed Description of the Invention, will be better understood when read in conjunction with the accompanying drawings.

FIG. 1 is a perspective view of a biodegradable stent graft, in accordance with aspects of the invention.

FIG. 2 is a perspective view of an alternative embodiment of a biodegradable stent frame, in accordance with aspects of the invention.

### DETAILED DESCRIPTION

The following discussion and accompanying figures disclose a biodegradable stent graft in accordance with various aspects of the present invention. Example embodiments of the stent graft is depicted in the figures and discussed below as having a configuration that is suitable for use in human vessels. The concepts disclosed with respect to human vessels may, however, be applied to any non-human vessel or other flexible tubular structure for a wide range of other utilities, including veterinary applications, for example, and may also be applied to various non-medical (non-health related) uses. Accordingly, one skilled in the relevant art will recognize that the concepts disclosed herein may have a wide range of applications and are not limited to the specific embodiments discussed below and depicted in the figures.

In general, and according to an embodiment, a stent graft is provided for reconstructing tissue in a vessel may include a biodegradable frame and a sheet containing a biological material. The sheet coats the biodegradable frame. The sheet may be capable of flexing in unison with the frame. The frame and the sheet may be capable of flexing in a radial direction to at least partially conform to at least a portion of a wall of the vessel.

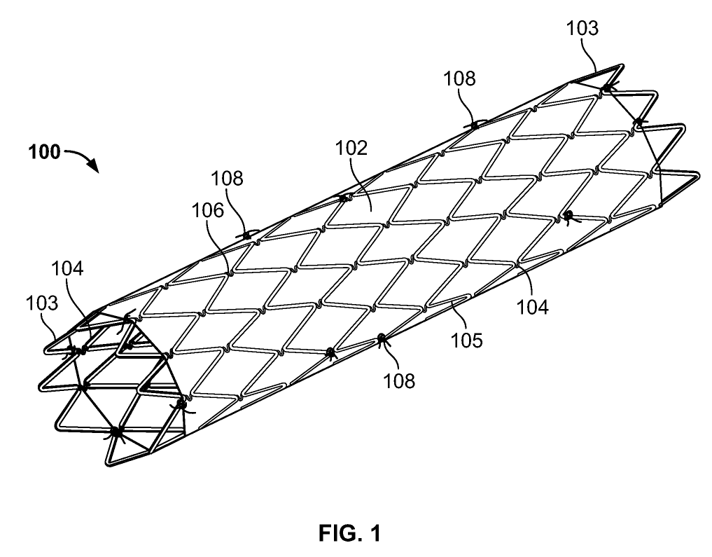

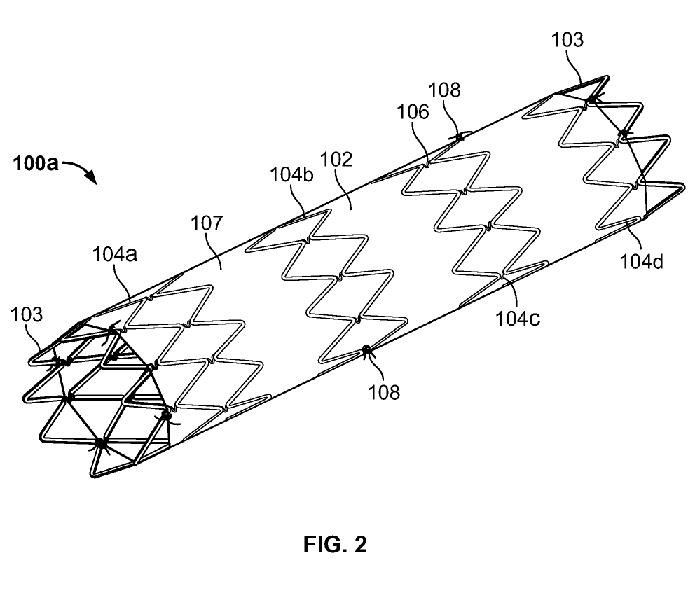

FIG. 1 illustrates a first arrangement of a stent graft 100. The stent graft 100 includes a biodegradable frame 104 and a sheet 102. The sheet 102 surrounds and may coat the biodegradable frame 104 throughout at least a significant portion of the longitudinal length of the biodegradable frame 100. In the depicted embodiments of FIG. 1 and 2, the sheet 102 does not extend to the longitudinal ends of the frame 104 and therefore leaves the end sections 103 of the frame 104 exposed. However, the sheet 102 can extend to the ends of the frame if desired. As assembled, the biodegradable frame 104 and the sheet 102 are capable of flexing in a radial direction to at least partially conform to at least a portion of a wall of a vessel to be repaired.

It is understood that the biodegradable frame 104 may have the properties of any desired stent structure. Additionally, the biodegradable frame 104 is tubular shaped and is capable of sufficient flexing under desired conditions. In a first arrangement, as shown, the biodegradable frame 104 is made from an expandable wire form. In an alternate arrangement, the biodegradable frame is made from a perforated tube. If a wire form design is used, as shown, any desirable wire form configuration may be used. For example, as shown, the wire form may be made from a series of circumferential frame elements 105 that are longitudinally joined together by joining members 106. According to this arrangement, the frame elements 105 are more flexible than the joining members 106 along the longitudinal direction of the stent graft 100.

The biodegradable frame 104 may be made of any suitable material. For example, in a first embodiment, the frame 104 is made from a magnesium alloy. In a second embodiment, the frame 104 is made from a poly-lactide which is a biodegradable, thermoplastic, aliphatic polyester derived from renewable resources, such as corn starch. In another embodiment, the frame 104 is made from an iron alloy. Biodegradable magnesium stents and poly-lactide stents are known in art and have been used as unshrouded devices to treat blockages in coronary arteries. By biodegradable, as used herein, it is meant that the frame substantially dissolves into small pieces, loses its shape, and is substantially carried off in the blood stream. In the blood stream, and based on the composition of the frame 104, the broken off molecular sized pieces are hydrolyzed and filtered according to the body's normal processes. The biodegradable frame 104 is biodegradable in a blood vessel of an average human under standard conditions in a period of 5 years or less, 1 year or less, and/or 6 months or less based on the composition of the frame 104.

The sheet 102 is preferably made from materials and is configured to be incorporated into the natural tissue such that a vessel wall will grow into it. That is, the sheet 102 will be bioabsorbed in the patient. The sheet 102 is configured such that the vessel wall will fully grow into it under standard conditions in a period of 1 year or less, 6 months or less, and/or 2 months or less based on the material properties of the sheet 102. In one arrangement, the sheet 102 is collagen-based such as a protein-based biologic collagen matrix. In another arrangement, the sheet may be an acellular dermal matrix. Alternatively, the sheet material can be derived from a biologic source, such as a pig intestine. In other embodiments, the sheet 102 may be made of any material suitable for tissue reconstruction including donated human skin, which may be skin from the patient himself. Accordingly, under such an approach, the skin would be taken from elsewhere on the patient's body and applied to outside of the frame 104. The frame 104/sheet 102 combination would be utilized as described below.

In a first embodiment, the outer surface of the sheet 102 is adhesive free and the vessel wall will grow into it aided by the force applied to it from the frame 104 once deployed. The sheet 102, and more specifically the outer surface of the sheet 102, may include seeding cells (not shown) coupled to it. The seeding cells may be endothelial cells or stem cells from the patient or a matching donor. The seeding cells help promote cell ingrowth from the inner vessel wall to the sheet 102.

The sheet 102 is attached to the frame 104 in any desirable manner. In a first arrangement, the sheet 102 is sewn to the frame 104 at selected points along the length and circumference of the frame 104 based on the design of the frame 104. Element 108 depicts sewing points 108 between the sheet 102 and the frame 104. Alternatively, or in addition, the sheet 102 may be joined to the frame 104 by suitable body-compatible adhesives such as fibrin glue. In a third embodiment, not shown, the stent graft has a sheet inside the frame in addition to the outer sheet 102. The inner and outer sheets are compressed or are heat sealed with the frame 104 therebetween.

FIG. 2 depicts an alternative embodiment to FIG. 1. More specifically, the embodiment of FIG. 2 differs from that of FIG. 1, in that the frame is not a unitary element. Rather, the frame is constructed of at least two longitudinally spaced independent frame sections. In the depicted embodiment of FIG. 2, the frame is formed from at least four frame sections 104a, 104b, 104c, and 104d. Flexibility along the longitudinal axis is enhanced by the gap sections 107 of the stent graft 100a where there is a circumferential gap in the frame as the flexibility is based on the characteristics of the sheet 102.

The biodegradable stent graft 100, 100a may be used to perform a repair to any desired vessel such as an artery or a vein. More specifically, the biodegradable stent graft 100, 100a, can be used to deliver a sheet or layer of biological material to a location in a vessel or other tubular structure for tissue ingrowth. The vessel that can be repaired with the biodegradable stent graft 100 can be a vessel in any desired location of a body, including, but not limited to, arms, shoulders, legs, a chest, a neck, and an abdomen. Particular vessels that would gain benefit from using the biodegradable stent graft 100, 100a include, but are not limited to the distal subclavian artery, the brachial artery, the axillary artery, the proximal femoral artery, the popliteal artery, the carotid artery, and the iliac artery. Additional benefits can also be obtain by use in repairing vessels that tend to be mobile and/or vessels that extend through or across at least a portion of a joint.

Like known deployment approaches for stents, the biodegradable stent graft 100, 100a may be designed to be expanded by the dilation of a balloon catheter. Alternatively, the frame 104 may be designed to have properties to be self-expandable. By way of example, the deployment of the biodegradable stent graft 100, 100a is described below as if performed a balloon catheter. This balloon catheter deployment process includes steps similar to existing methods for deploying a permanent stent with a balloon catheter.

In one procedure method, the first end of guide wire may be inserted into a femoral artery. It can then be maneuvered through the vessels in the body to and past the location of the damaged portion of the desired vessel. The balloon catheter (in a deflated state) is guided over the guide wire. The balloon catheter preferably has the biodegradable stent graft 100, 100a positioned on it so it can be centered at the location of the damaged portion of the vessel. The balloon catheter is inflated from outside the patient's body at or near the opposing end of the guide wire. The biodegradable stent graft 100, 100a expands in the radial direction due to the inflation of the balloon. This therefore results in the biodegradable stent graft 100, 100a expanding at the site of the damaged vessel. The sheet 102 adheres to the interior surface of the vessel wall and remains in the expanded state due to the geometry and properties of the frame 104. The balloon catheter may be deflated and removed. The biodegradable stent graft 100 remains in the vessel at the site of damage and supports and patches the vessel wall.

Based on the materials used, over a period of time, such as 5 years or less, 1 year or less, or 6 months or less, the frame 104 biodegrades into small pieces and is substantially carried off in the blood stream. In the blood stream, and based on the composition of the frame 104, the broken off molecular sized pieces are hydrolyzed and filtered in the body's normal processes. The sheet 102 may be bioabsorbed in the vessel wall within preferably shorter limits of time such as a year or less, 6 months or less, or 2 months or less. Effectively, the frame 104 serves as a delivery system for the grafting sheet 102. It initially supports the sheet 102 and applies a radial force to aid in the ingrowth. Over time, the after the ingrowth is effected, the frame 104 will biodegrade in the patient and the sheet 102 will be bioabsorbed. This ends up leaving an ideal result—a permanently patched vessel wall with a bioabsorbed patch and no residual stenting structure. This also eliminates situations of permanent stresses and strains to the repaired vessel. The biodegradability and the bioabsorption of the stent graft 100, 100a also provides other advantages such as enabling the endovascular therapy rather than more invasive alternatives when a patient has a damaged vessel in a location that is subject to a high degree of movement such as at or near a joint. This type of endovascular therapy would result in a lower surgical risk for the patient, and a faster recovery time.

The above discussion details the structure and configuration of biodegradable stent grafts, as depicted in the figures. Various modifications may be made to these biodegradable stent grafts without departing from the intended scope of the present invention. For example, the biodegradable frame and the sheet may be made of any suitable material that may not be currently known in the art.

The present invention is disclosed above and in the accompanying drawings with reference to a variety of embodiments. The purpose served by the disclosure, however, is to provide an example of the various features and concepts related to the invention, not to limit the scope of the invention. One skilled in the relevant art will recognize that numerous variations and modifications may be made to the embodiments described above without departing from the scope of the present invention, as defined by the appended claims.

That which is claimed is:

1. A device for reconstructing tissue in a vessel, comprising:
a biodegradable frame; and
a sheet containing a biological material and configured to enable the absorption of the sheet by a blood vessel, the sheet coating the biodegradable frame and capable of flexing in unison with the biodegradable frame;
wherein the biodegradable frame and the sheet are capable of flexing in a radial direction for positioning the sheet adjacent a portion of the wall of the vessel.

2. The device of claim 1, wherein the frame is shaped to be a web comprising a plurality of expandable elements.

3. The device of claim 2, wherein the plurality of expandable elements comprises a first expandable element and a second expandable element that is capable of flexing independently of the first expandable element.

4. The device of claim 1, wherein the biodegradable frame and the sheet form a flexible tube and are configured to expand to conform to the shape of the wall of the vessel.

5. The device of claim 1, wherein the biodegradable frame includes magnesium.

6. The device of claim 1, wherein the biodegradable frame is made from a poly-lactide.

7. The device of claim 1, wherein the biodegradable frame is made from materials configured to be biodegradable in a blood vessel of a human under standard conditions in a period of 5 years or less, and the sheet is made from materials configured to be absorbed by the wall of the vessel in a period of 1 year or less.

8. The device of claim 7, wherein the biodegradable frame is made from materials configured to be biodegradable in a blood vessel of a human under standard conditions in a period of 1 year or less, and the sheet is made from materials configured to be absorbed by the wall of the vessel in a period of 6 months or less.

9. The device of claim 8, wherein the biodegradable frame is made from materials configured to be biodegradable in a blood vessel of a human under standard conditions in a period of 6 months or less, and the sheet is made from materials configured to be absorbed by within the wall of the vessel in a period of 2 months or less.

10. The device of claim 1, wherein the sheet is a biologic arterial graft.

11. The device of claim 1, wherein the sheet is an acellular dermal matrix.

12. The device of claim 1, wherein the biodegradable frame is made from materials configured to be biodegradable in a blood vessel of a human under standard conditions in a period of 6 months or less, and the sheet is made from materials configured to be absorbed by the wall of the vessel in a period of 6 months or less, the biodegradable frame includes at least one of a poly-lactide and magnesium, and wherein the sheet is one of a biologic arterial graft and an acellular dermal matrix.

13. The device of claim 1, further comprising seeding cells attached to the sheet to promote the cell ingrowth from the vessel wall.

14. The stent graft of claim 13, wherein the seeding cells include at least one of endothelial cells or stem cells.

15. The stent graft of claim 1, wherein the frame includes a plurality of distinct, longitudinally separated, radial expandable frame elements, with each frame element being coupled to the sheet.

16. A method of reconstructing tissue in a vessel, comprising steps of:
positioning a delivery device at a site of damage in a vessel of a patient, the delivery device including a biodegradable frame and a sheet containing a biological material, the sheet coating the biodegradable frame;
affixing the delivery device to a wall of the vessel at the site of damage; and
leaving the delivery device in the vessel and permitting the biodegradable frame to dissolve and be carried off in the blood stream.

17. The method of claim 17, wherein the step of leaving includes permitting the sheet to biologically integrate with the vessel wall.

18. The method of claim 17, wherein the step of positioning the delivery device includes positioning the delivery device at a joint of the patient.

19. The method of claim 17, wherein the step of positioning the delivery device includes positioning the delivery device in a vessel of one of an arm, a leg, the chest, the neck, and the abdomen of the patient.

20. The method of claim 17, further comprising the step of removing skin from the patient to form at least part of the sheet, and covering the frame with the removed skin.
